(19) United States
(12) Patent Application Publication
(10) Pub. No.: US 2009/0299119 A1
(43) Pub. Date: Dec. 3, 2009

(54) Heat Balanced FCC For Light Hydrocarbon Feeds

(75) Inventors: Alan Claude, Houston, TX (US); Anand Subramanian, Sugar Land, TX (US)

Correspondence Address: KELLOGG BROWN & ROOT LLC;ATTN: Christian Heausler, 4100 Clinton Drive, HOUSTON, TX, 77020, US

(73) Assignee: KELLOGG BROWN & ROOT LLC, Houston, TX (US)

(21) Appl. No.: 12/128,971

(22) Filed: May 29, 2008

(51) Int. Cl. C07C 4/02

(52) U.S. Cl. 585/653

(57) ABSTRACT

Systems and methods for processing hydrocarbons are provided. A first hydrocarbon feed containing one or more C4 to C20 hydrocarbons having a research octane number of less than 88 can be cracked at a first temperature and in the presence of one or more catalysts to provide a first cracked mixture. A second hydrocarbon feed can be introduced to the first mixture to provide a second mixture. The second mixture can be cracked at the first temperature to provide a second cracked mixture containing propylene and one or more hydrocarbons having a research octane number of about 88 to about 95, and one or more coked catalysts.

### BACKGROUND

1. Field

The present embodiments generally relate to systems and methods for processing hydrocarbons. More particularly, embodiments of the present invention relate to systems and methods for thermally balancing an FCC riser reactor.

2. Description of the Related Art

Fluid catalytic crackers (FCC) are a mainstay in the conversion of raw hydrocarbons into one or more preferred products. An FCC consists of few components: one or more riser reactors, one or more disengagers and one or more regenerators. A hydrocarbon feed and one or more catalysts are added to the riser reactor which is maintained at an elevated temperature and/or pressure. The cracking of the hydrocarbons within the riser reactor produces one or more cracked hydrocarbons and small quantities carbonaceous coke which is deposited on the surface of the catalyst. These coke deposits deactivate the catalyst after passage through the riser reactor. The cracked hydrocarbons and the coked catalyst exit the riser reactor and are introduced to one or more disengagers where the coked catalyst is separated from the cracked hydrocarbons. The cracked hydrocarbons are removed from the FCC for further processing and/or treatment. The coked catalyst is introduced to one or more regenerators where the coke is combusted, oxidized, and/or converted to one or more waste gases. The combustion process removes the coke from the surface of the catalyst, regenerating the catalyst, and permitting its recycle back to the riser reactor. Maintaining high temperatures throughout the FCC and ensuring sufficient coke build on the catalyst to provide the heat necessary to regenerate the catalyst are therefore of primary importance in the operation of an FCC.

Light hydrocarbon feeds, those containing twelve or fewer carbon atoms, can be used as a feed to an FCC producing olefinic hydrocarbons such as ethylene and propylene. The production of olefins using an FCC generally requires the use of specialized catalysts. Additionally, the FCC must be operated at high severity, i.e. high temperatures and/or pressure, to promote the cracking of the feed into desired olefinic hydrocarbons. Due to the high temperature required and the relatively low heating value of the light hydrocarbon feed, FCCs producing olefins typically require a supplemental fuel source to maintain riser reactor temperature and to regenerate the catalyst within the regenerator.

Thus, a need exists for improved systems and methods for converting one or more light hydrocarbon feeds into olefinic products without requiring a supplemental fuel source within the regenerator.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts an illustrative system for upgrading one or more hydrocarbons according to one or more embodiments described herein.

FIG. 2 depicts another illustrative system for upgrading one or more hydrocarbons according to one or more embodiments described herein.

### DETAILED DESCRIPTION

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this patent is combined with available information and technology.

Systems and methods for processing hydrocarbons are provided. A first hydrocarbon feed containing one or more C₄ to C₂₀ hydrocarbons having a research octane number of less than 88 can be cracked at a first temperature and in the presence of one or more catalysts to provide a first cracked mixture. A second hydrocarbon feed can be introduced to the first mixture to provide a second mixture. The second mixture can be cracked at the first temperature to provide a second cracked mixture containing ethylene, propylene, one or more hydrocarbons having a research octane number of about 88 to about 95, and one or more coked catalysts

The one or more catalysts can include one or more cracking catalysts suitable for the production of olefins and one or more cracking catalysts suitable for improving the research octane number of the cracked hydrocarbon compounds in the first cracked mixture. The one or more catalysts can include mixtures containing both catalysts useful for increasing the production of olefins and catalysts useful for increasing the research octane number of the cracked hydrocarbon compounds in the second cracked mixture, catalytically cracking the heavier second feedstock into more useful lighter products, and promoting the formation of coke.

The second hydrocarbon feed can include one or more fuel gases, methane, hydrogen, ethane, propane, demetallized oils, deasphalted oils, vacuum gas oils, fuel oils, gas oils, resids having twenty or more carbon atoms (C₂₀+), mixtures thereof or any combination thereof. The combustion of the second hydrocarbon feed can deposit a layer of coke on the one or more catalysts. Subsequent combustion of the coke can heat the one or more catalysts prior to mixing the one or more catalysts with the first hydrocarbon feed.

The pressure, first temperature, and second temperature can be maintained at “high severity” conditions—i.e. at high temperatures, pressures and/or catalyst to oil ratios. High severity conditions can favor the cracking of the first hydrocarbon feed to one or more olefinic compounds, including ethylene, propylene. High severity conditions can also improve the octane rating of the cracked hydrocarbons. In one or more embodiments, the first temperature can be in the range of about 500° C. (930° F.) to about 700° C. (1,290° F.). In one or more embodiments, the second temperature can be lower than the first due to the cooling effects of heating and or vaporizing the second feed as well as the heat input required to sustain the cracking reactions. The coke formed by these cracking reactions can provide sufficient fuel value to maintain the higher first temperature. After separating the coke-covered catalyst from the one or more hydrocarbons, the coke can be combusted to raise the temperature of the catalyst prior to recycling and mixing with the first hydrocarbon feed. The feed rate of the second hydrocarbon feed can be maintained at a value consistent with producing a desired first temperature within the first mixture.

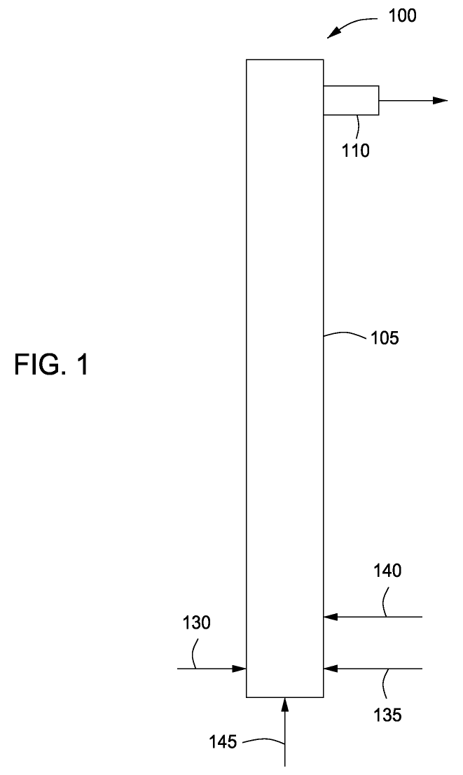

FIG. 1 depicts an illustrative system 100 for upgrading one or more hydrocarbons according to one or more embodiments. In one or more embodiments, the system 100 can include one or more riser reactors 105. The hydrocarbon feed (“first hydrocarbon feed”) in line 130 can be mixed or otherwise combined with one or more catalysts supplied via line 135, and optionally, steam supplied via line 145. The first hydrocarbon feed in line 130 can include, but is not limited to one or more hydrocarbon compounds having from four to twelve carbon atoms (C₄ to C₂₀). In one or more specific embodiments, the first hydrocarbon feed can include, but is not limited to one or more hydrocarbon compounds having from four to eight carbon atoms (C₄ to C₁₂). The first hydrocarbon feed can have a research octane number of from about 60 to about 88; about 65 to about 88; or about 70 to about 88. In one or more embodiments, the first hydrocarbon feed can have an olefin content from about 0% wt. to about 75% wt.; from about 0% wt. to about 50% wt.; or from about 0% wt. to about 25% wt. The first hydrocarbon feed can have a normal boiling point of from about 95° C. (200° F.) to about 260° C. (500° F.); about 120° C. (250° F.) to about 240° C. (465° F.); or about 150° C. (300° F.) to about 220° C. (430° F.).

The first hydrocarbon feed in line 130 can be partially or completely vaporized prior to introduction to the riser reactor 105. In one or more embodiments, the first hydrocarbon feed in line 130 can be about 25% wt. or more; about 50% wt. or more; about 75% wt. or more; about 90% wt. or more; about 95% wt. or more; about 99% wt. or more; or about 99.9% wt. or more vaporized prior to introduction to the riser reactor 105. The first hydrocarbon feed in line 130 can be introduced to the riser reactor 105 at ambient or elevated temperature. In one or more embodiments, the temperature of the first hydrocarbon feed in line 130 can be a minimum of about 40° C. (105° F.); about 100° C. (212° F.); about 200° C. (390° F.); about 400° C. (750° F.); or about 500° C. (930° F.).

The optional steam introduced via line 145 can be either saturated or superheated. In one or more embodiments, the steam introduced via line 145 can be saturated, having a minimum supply pressure of about 135 kPa (5 psig); about 310 kPa (30 psig); about 510 kPa (60 psig); about 720 kPa (90 psig); about 1,130 kPa (150 psig); or about 2,160 kPa (300 psig). In one or more embodiments, the steam introduced via line 145 can be superheated having a minimum superheat of about 15° C. (30° F.); about 30° C. (60° F.); about 45° C. (90° F.); about 60° C. (120° F.; or about 90° C. (150° F.).

The one or more catalysts supplied via line 135 can include catalysts suitable for catalytically cracking the first and second hydrocarbon feeds to provide one or more olefinic hydrocarbons and/or one or more mixed hydrocarbons suitable for blending into one or more fungible products including, but not limited to one or more olefins, one or more paraffins, one or more naphthenes, one or more aromatics or any combination thereof. The one or more catalysts can include, but are not limited to, one or more of the following: ZSM-11, ZSM-12, ZSM-23, ZSM-35, ZSM-38, Y-type zeolites, metal impregnated catalysts, zeolites, faujasite zeolites, modified faujasite zeolites, Y-type zeolites, ultrastable Y-type zeolites (USY), rare earth exchanged Y-type zeolites (REY), rare earth exchanged ultrastable Y-type zeolites (REUSY), rare earth free Z-21, Socony Mobil #5 zeolite (ZSM-5), high activity zeolite catalysts, mixtures thereof or combinations thereof.

The catalyst supplied via line 135 can be introduced to the riser reactor 105 at a rate proportionate to the first hydrocarbon feed, the second hydrocarbon feed, or the combined first and second hydrocarbon feeds. In one or more embodiments, the catalyst feed-to-hydrocarbon feed weight ratio can range from a minimum of about 4:1; about 8:1; or about 12:1 to a maximum of about 18:1; about 25;1; about 30:1; or about 70:1. In one or more specific embodiments the catalyst feed-to-hydrocarbon feed weight ratio in the riser reactor 105 can be about 4:1 to about 30:1. In one or more embodiments, the one or more catalysts can be introduced to the riser reactor at a temperature above the temperature of the first catalyst/hydrocarbon mixture to provide heat for increasing the feed temperature to final reaction temperature and to sustain the endothermic cracking reactions. In one or more embodiments, the temperature of the catalyst can range from a minimum of about 600° C. (1,110° F.); about 650° C. (1,200° F.); or about 700° C. (1,290° F.) to a maximum of about 785° C. (1,445° F.); about 815° C. (1,500° F.); or about 850° C. (1,560° F.). The combined first hydrocarbon feed and one or more catalysts (“first mixture”) can be maintained at a temperature (“first temperature”) of from about 300° C. (570° F.) to about 900° C. (1,650° F.); about 400° C. (750° F.) to about 800° C. (1,470° F.); or about 500° C. (930° F.) to about 700° C. (1,290° F.).

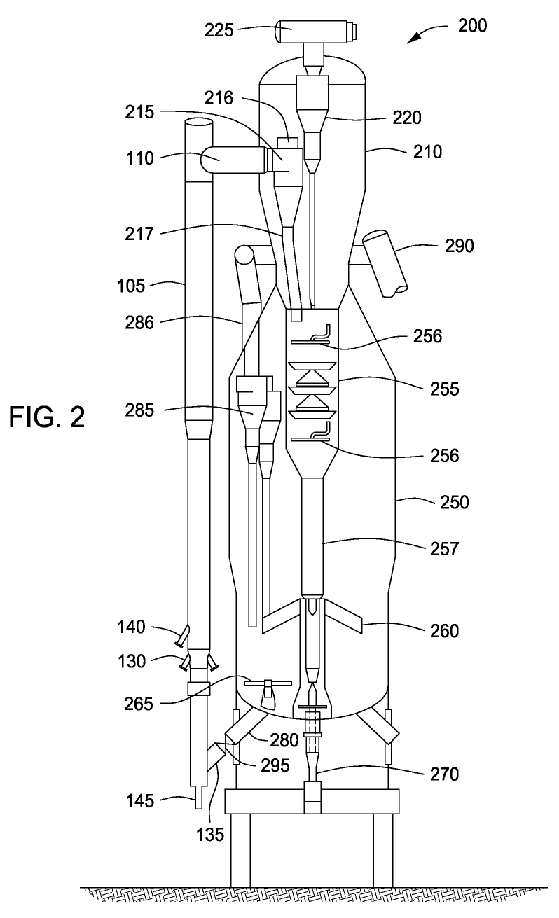

The riser reactor 105 can include any system, device or combination of systems or devices suitable for the cracking of one or more hydrocarbon feeds in the presence of one or more catalysts. The riser reactor 105 can be a riser on a fluidized catalytic cracker (“FCC”) described in greater detail with reference to FIG. 2. The riser reactor 105 can be configured in any physical orientation or geometry, including horizontal (0° elevation), vertical (90° elevation) including any intermediate angle therebetween. The riser reactor 105 can operate at a temperature of from about 200° C. (390° F.) to about 1,700° C. (3,090° F.); about 300° C. (570° F.) to about 1,400° C. (2,550° F.); about 400° C. (750° F.) to about 1,000° C. (1,830° F.); or about 500° C. (930° F.) to about 700° C. (1,290° F.). The riser reactor 105 can operate at a pressure of from about 140 kPa (5 psig) to about 2,160 kPa (300 psig); about 140 kPa (5 psig) to about 1,130 kPa (150 psig); or from about 140 kPa (5 psig) to about 720 kPa (90 psig).

The second hydrocarbon feed, supplied via line 140 can be introduced at any point within the riser reactor 105. In one or more embodiments, the second hydrocarbon feed in line 140 can be introduced at same point as the first hydrocarbon feed in line 130. In one or more embodiments, the second hydrocarbon feed in line 140 can be introduced at subsequent point to the first hydrocarbon feed in line 130. In one or more embodiments, the second hydrocarbon feed in line 140 can be introduced simultaneously, sequentially, alternatively or in any other manner or frequency in relation to the first hydrocarbon feed in line 130.

The second hydrocarbon feed in line 140 can be partially or completely vaporized prior to introduction to the riser reactor 105. In one or more embodiments, the second hydrocarbon feed in line 140 can be about 5% wt. or more; about 10% wt. or more; about 25% wt. or more; about 50% wt. or more; about 75% wt. or more; about 90% wt. or more; or about 99.9% wt. or more vaporized prior to introduction to the riser reactor 105. The second hydrocarbon feed in line 140 can be introduced to the riser reactor at ambient or elevated temperature. In one or more embodiments, the second hydrocarbon feed in line 140 can be at a temperature of about 40° C. (105° F.) or more; about 100° C. (212° F.) or more; about 200° C. (390° F.) or more; or about 370° C. (700° F.) or more.

The fuel or coke producing value of the second hydrocarbon feed in line 140 can be sufficient to maintain the desired temperature within the riser reactor 105. In one or more embodiments, the temperature of the catalyst introduced to the riser reactor can be adjusted by varying the deposition of coke on the surface of the catalyst. In one or more embodiments, the quantity of coke deposited on the catalyst can be adjusted by varying the second hydrocarbon feed to the reactor. In one or more embodiments, the pressure and second temperature within the riser reactor 105 can be maintained at “high severity” conditions. In one or more embodiments, the first and second temperatures can be substantially similar, i.e. having a difference less than 55° C. (100° F.), or different, i.e. having a difference greater than 55° C. (100° F.). Operation of the riser reactor 105 at high severity conditions throughout can favor the cracking of the first and second hydrocarbon feeds into one or more olefinic compounds, such as ethylene and/or propylene, and one or more mixed hydrocarbons having a research octane number suitable for fuels blending.

Within the riser reactor 105, the first hydrocarbon feed and second hydrocarbon feed, and the one or more catalysts can crack, react, convert, and/or otherwise recombine to provide a mixture containing one or more cracked hydrocarbons (“second cracked mixture”). As the hydrocarbons present in the riser reactor 105 crack and decompose to form finished products, at least a portion of the first and second hydrocarbon feeds can deposit as a layer of carbonaceous coke on the exterior surface of the one or more catalysts. The deposition of coke on the surface of the catalyst deactivates the catalyst and forms coke-covered catalyst. The coke-covered catalyst can exit the riser reactor 105 suspended in the second cracked mixture in line 110.

The hydrocarbons present in the second cracked mixture in line 110 can include one or more olefinic hydrocarbons, such as ethylene and propylene, and one or more mixed hydrocarbons. The one or more mixed hydrocarbons present in the second cracked mixture in line 110 can have a research octane number greater than the research octane number of the first hydrocarbon feed in line 130.

In one or more embodiments, the second cracked mixture in line 110 can have an ethylene concentration of from about 0.1% vol. to about 20% vol.; about 0.5% vol. to about 17% vol.; or from about 1% vol. to about 15% vol. In one or more embodiments, the second cracked mixture in line 110 can have a propylene concentration of from about 0.1% vol. to about 25% vol.; about 0.5% vol. to about 17% vol.; or from about 1% vol. to about 15% vol. In one or more embodiments, in the second cracked mixture in line 110 can have a mixed hydrocarbons concentration of from about 1% vol. to about 75% vol.; about 5% vol. to about 40% vol.; or from about 5% vol. to about 30% vol. In one or more embodiments, the second cracked mixture in line 110 can have a solids concentration of from about 500 ppmw to about 98% wt.; about 2,500 ppmw to about 75% wt.; about 1% wt. to about 50% wt.; or from about 5% wt. to about 50% wt.

In one or more embodiments, all or a portion of the mixed hydrocarbons in the second cracked mixture in line 110 can be used to provide a high octane gasoline blendstock. In one or more embodiments, the one or more mixed hydrocarbons in the second cracked mixture in line 110 can have a research octane number of from about 88 to about 100; about 88 to about 97; or about 88 to about 92. In one or more embodiments, the mixed hydrocarbons in the second cracked mixture can have a bulk normal boiling point of from about 95° C. (200° F.) to about 260° C. (500° F.); about 120° C. (250° F.) to about 240° C. (465° F.); or about 150° C. (300° F.) to about 220° C. (430° F.).

FIG. 2 depicts another illustrative system 200 for upgrading one or more hydrocarbons according to one or more embodiments described. The system 200 includes one or more riser reactors 100, one or more disengagers 210, and one or more regenerators 250. The one or more disengagers 200 can include one or more riser cyclones 215, upper cyclones 220, plenums 225, catalyst strippers 255, catalyst distributors 260, and plug valves 270. The one or more regenerators 250 can include one or more air distributors 265, regenerator cyclones 285, and plenums 290. In one or more embodiments, the system 200 can include, but is not limited to one or more fluidized catalytic cracking systems 200 depicted in FIG. 2.

The second cracked mixture exiting the riser reactor 105 via line 110 can be introduced to the one or more disengagers 200. Within the disengager 200, the second cracked mixture can flow into the one or more riser cyclones 215 wherein at least a portion of the coke-covered catalyst can be selectively separated from the second cracked mixture. The second cracked mixture can exit the one or more riser cyclones 215, flowing into the one or more upper cyclones 220 wherein additional coke-covered catalyst can be separated.

The near solids-free second cracked mixture can flow from the one or more upper cyclones 220 into the one or more disengager plenums 225 for withdrawal and subsequent fractionation or separation into one or more finished hydrocarbon products, for example propylene and one or more mixed hydrocarbons. The near-solids free cracked second mixture in the one or more disengager plenums 225 can have a solids concentration of from about 5 ppmw to about 5% wt.; about 10 ppmw to about 4% wt.; about 25 ppmw to about 3.5% wt.; or from about 50 ppmw to about 3% wt.

The coke-covered catalyst separated from the second cracked mixture in the one or more riser cyclones 215 and the one or more upper cyclones 220 can be introduced to the one or more catalyst strippers 255. Within the one or more catalyst strippers 255, steam can be introduced into the coke-covered catalyst in the one or more catalyst strippers 255 using one or more steam distributors 256. The passage of steam through the catalyst stripper 255 can assist in removing any residual hydrocarbons entrained or entrapped within the coke-covered catalyst prior to regenerating the catalyst. The steam, carrying one or more hydrocarbons stripped from the coke-covered catalyst in the catalyst stripper 255, can flow into the disengager 210, thence into the one or more upper cyclones 220.

The steam supplied to the catalyst stripper 255 via the one or more distributors 256 can be saturated or superheated. In one or more embodiments, the steam introduced via the one or more distributors 256 can be saturated, having a minimum supply pressure of about 135 kPa (5 psig); about 310 kPa (30 psig); about 510 kPa (60 psig); about 720 kPa (90 psig); about 1,130 kPa (150 psig); or about 2,160 kPa (300 psig). In one or more embodiments, the steam introduced via the one or more distributors 256 can be superheated having a minimum superheat of about 15° C. (30° F.); about 30° C. (60° F.); about 45° C. (90° F.); about 60° C. (120° F.); or about 90° C. (150° F.).

Coke-covered catalyst can flow from the catalyst stripper 255 into a standpipe 257. A first portion of the coke-covered catalyst within the standpipe 257 can flow via one or more distributors 260 into one or more regenerators 250. The remaining portion of coke-covered catalyst within the standpipe 257 can be withdrawn from the system 200 via one or more plug valves 270. In one or more embodiments, about 5% wt.; about 10% wt.; about 25% wt.; about 50% wt.; about 75% wt.; about 85% wt.; about 90% wt.; about 95% wt.; or about 99% wt. of the coke-covered catalyst in the standpipe 257 can be introduced to the one or more regenerators 250 via the one or more distributors 260, with the balance withdrawn from the standpipe 257 via the one or more plug valves 270. At least a portion of fresh catalyst make-up can be added to the system 200 via addition to either the one or more riser reactors 105 and/or the regenerator 250.

Within the one or more regenerators 250 one or more oxidants can be distributed via one or more air distributors 265. The addition of air to the coke-covered catalyst discharged from the standpipe 257 can result in the oxidation and/or combustion of the coke on the surface of the catalyst into one or more waste gases including, but not limited to: carbon monoxide, carbon dioxide, hydrogen, water vapor, mixtures thereof or combinations thereof. The removal of the coke from the surface of the catalyst can re-expose the surface of the catalyst, thereby reactivating and/or regenerating the catalyst. At least a portion of the reactivated and/or regenerated catalyst can be recycled from the regenerator 250 to the one or more riser reactors 105 via one or more standpipes 280, valves 295 discharging into line 135.

As used herein, the term “oxidants” can refer to any compound or element suitable for oxidizing the coke on the surface of the catalyst. Such oxidants can include, but are not limited to, air, oxygen enriched air (air having an oxygen concentration greater than 21% wt.), oxygen, or nitrogen enriched air (air having a nitrogen concentration greater than 79% wt.).

At least a portion of the regenerated catalyst can flow via one or more standpipes 280 to line 135 for recycle to one or more riser reactors 105. The flow of regenerated catalyst from the regenerator 250 to the riser reactors 105 can be controlled using one or more valves 295. Regenerated catalyst can fulfill at least a portion of the catalyst requirement of the one or more riser reactors 105. In one or more embodiments, a minimum of about 25% wt.; about 50% wt.; about 75% wt.; about 85% wt.; about 90% wt.; about 95% wt. of the catalyst requirement in the one or more riser reactors 105 can be supplied using regenerated catalyst from the regenerator 250.

The one or more waste gases generated by the oxidation and/or combustion of the coke on the surface of the catalyst in the regenerator 250 can flow into the one or more regenerator cyclones 285 wherein at least a portion of the catalyst suspended in the waste gases can be removed and returned to the regenerator 250. Waste gases from the regenerator cyclones 285 can exit the regenerator via one or more ducts 286. The waste gases can be collected in the regenerator plenum 290. The collected waste gases in the regenerator plenum 290 can be directed for subsequent recovery, reuse, recycle, treatment, and/or disposal.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1) A method for processing hydrocarbons comprising:
mixing a first hydrocarbon feed comprising one or more C4 to C20 hydrocarbons with one or more catalysts to provide a first mixture at a first temperature, wherein the first hydrocarbon feed has a research octane number of less than 88;
introducing a second hydrocarbon feed to the first mixture to provide a second mixture; and
cracking the second mixture at a second temperature to provide a second cracked mixture comprising propylene, one or more hydrocarbons having a research octane number of about 88 to about 95, and one or more coked catalysts.

2) The method of claim 1, further comprising selectively separating the second cracked mixture to provide one or more finished products comprising propylene and one or more mixed hydrocarbons.

3) The method of claim 1, wherein at least a portion of the first mixture is cracked at the first temperature prior to adding the second hydrocarbon feed.

4) The method of claim 1, wherein the first temperature is about 500° C. to about 700° C.

5) The method of claim 1, wherein the difference between the first and second temperatures is less than 165° C.

6) The method of claim 1, wherein the difference between the first and second temperatures is greater than 165° C.

7) The method of claim 1, wherein the second hydrocarbon feed comprises hydrogen, methane, ethane, propane, demetallized oils, deasphalted oils, vacuum gas oils, fuel oils, gas oils, resids having twenty or more carbon atoms (C20+), mixtures thereof or any combination thereof.

8) The method of claim 1, wherein the one or more catalysts comprise ZSM-11, ZSM-12, ZSM-23, ZSM-35, ZSM-38, Y-type zeolites, metal impregnated catalysts, zeolites, faujasite zeolites, modified faujasite zeolites, Y-type zeolites, ultrastable Y-type zeolites (USY), rare earth exchanged Y-type zeolites (REY), rare earth exchanged ultrastable Y-type zeolites (REUSY), rare earth free Z-21, Socony Mobil #5 zeolite (ZSM-5), or high activity zeolite catalysts.

9) The method of claim 1, wherein the catalyst to hydrocarbon weight ratio is about 4:1 to about 30:1.

10) A method for processing hydrocarbons comprising:
cracking, at a first temperature, a first hydrocarbon feed comprising one or more C4 to C20 hydrocarbons in the presence of one or more catalysts to provide a first mixture, wherein the first temperature is about 500° C. to about 700° C. and wherein the first hydrocarbon feed has a research octane number of less than 88;
introducing a second hydrocarbon feed to the first mixture to provide a second mixture; and
cracking the second mixture, at a second temperature, to provide a second cracked mixture comprising propylene, one or more hydrocarbons having a research octane number of about 88 to about 95, and one or more coked catalysts, and wherein the second temperature is about 480° C. to about 650° C.

11) The method of claim 10, further comprising selectively separating the second cracked mixture to provide one or more finished products.

12) The method of claim 10, wherein the second hydrocarbon feed comprises hydrogen, methane, ethane, or propane.

13) The method of claim 10, wherein the second hydrocarbon feed comprises hydrogen, methane, ethane, propane, demetallized oils, deasphalted oils, vacuum gas oils, fuel oils, gas oils, resids having twenty or more carbon atoms (C20+), mixtures thereof or any combination thereof.

14) The method of claim 10, wherein the one or more catalysts comprise ZSM-11, ZSM-12, ZSM-23, ZSM-35, ZSM-38, Y-type zeolites, metal impregnated catalysts, zeolites, faujasite zeolites, modified faujasite zeolites, Y-type zeolites, ultrastable Y-type zeolites (USY), rare earth exchanged Y-type zeolites (REY), rare earth exchanged ultrastable Y-type zeolites (REUSY), rare earth free Z-21, Socony Mobil #5 zeolite (ZSM-5), or high activity zeolite catalysts.

15) The method of claim 10, wherein the catalyst to first hydrocarbon feed ratio is about 4:1 to about 30:1.

16) A system for processing hydrocarbons comprising:
a cracking zone wherein a first hydrocarbon feed comprising one or more C4 to C20 hydrocarbons is cracked at a first temperature in the presence of one or more catalysts to provide a first cracked mixture, and wherein the first hydrocarbon feed has a research octane number of less than 88;
a feed zone wherein a second hydrocarbon feed is mixed with the first cracked mixture to provide a second mixture; and
a cracking zone wherein the second mixture is cracked at the first temperature to provide a second cracked mixture comprising propylene, one or more hydrocarbons having a research octane number of about 88 to about 95, and one or more coked catalysts.

17) The system of claim 16, further comprising a separation zone wherein the second cracked mixture is selectively separated to provide one or more finished products comprising propylene and one or more hydrocarbons having a research octane number of about 88 to about 95.

18) The system of claim 16, further comprising at least one regeneration zone wherein the one or more coked catalysts can be regenerated to provide at least a portion of the one or more catalysts.

19) The system of claim 16, wherein the first temperature is about 500° C. to about 700° C.

20) The system of claim 16, wherein the first hydrocarbon feed is cracked at a pressure of about 5 psig to about 50 psig.
